(19) United States
(12) Patent Application Publication
(10) Pub. No.: US 2004/0210300 A1
(43) Pub. Date: Oct. 21, 2004

(54) Apparatus and method for maintaining flow through a vessel or duct

(76) Inventor: Walid Najib Aboul-Hosn, Fair Oaks, CA (US)

Correspondence Address: Jonathan Spangler, c/o PortofolioIP, P. O Box 52050, Minneapolis, MN, 55402, US

(21) Appl. No.: 10/644,599

(22) Filed: Aug. 19, 2003

Related U.S. Application Data
(63) Continuation of application No. PCT/US02/01845, filed on Jan. 19, 2002.

(51) Int. Cl. A61F 2/06

(52) U.S. Cl. 623/1.23; 623/1.36

(57) ABSTRACT

The present disclosure involves devices and related methods involving the use of a graft conduit (autologous or synthetic) which, when deployed within a diseased or occluded vessel or duct, reestablishes sufficient flow therethrough and isolates the diseased or occluded region from the rest of the vessels by forming a lining along the interior surface of the diseased or occluded region.

### BACKGROUND OF THE INVENTION

I. Field of the Invention

The present invention relates generally to structures or devices for implantation in a body to maintain the lumen of a duct or vessel open for unimpeded passage of liquid, solid or gas therethrough. More particularly, the present invention relates to a graft assembly having a length of graft conduit (autologous or synthetic) which, when deployed within a diseased or occluded vessel or duct, reestablishes sufficient flow therethrough and isolates the diseased or occluded region from the rest of the vessel by forming a lining along the interior surface of the diseased or occluded region.

II. Discussion of the Prior Art

Vascular stenosis is a major problem in health care worldwide, and is characterized as the narrowing (and potential blocking) of blood vessels as a result of the deposition of fatty materials, cellular debris, calcium, and/or blood clots (collectively referred to as “flow restrictions”). Current treatments to overcome flow restrictions include the administration of thombolytics (clot-dissolving drugs), interventional devices, and/or bypass surgery. As will be demonstrated below, these state-of-the-art techniques and devices all fail to adequately answer the vexing problem of maintaining blood flow through blood vessels.

Thromolytics are typically administered in high doses. However, even with aggressive therapy, thrombolytics fail to restore blood flow in the affected vessel in about 30% of patients. In addition, these drugs can also dissolve beneficial clots or injure healthy tissue causing potentially fatal bleeding complications.

Interventional procedures include angioplasty, atherectomy, and laser ablation. However, the use of such devices to remove flow-restricting deposits may leave behind a wound that heals by forming a scar. The scar itself may eventually become a serious obstruction in the blood vessel (a process known as restenosis). Also, diseased blood vessels being treated with interventional devices sometimes develop vasoconstriction (elastic recoil), a process by which spasms or abrupt reclosures of the vessel occur, thereby restricting the flow of blood and necessitating further intervention. Approximately 40% of treated patients require additional treatment for restenosis resulting from scar formation occurring over a relatively long period, typically 4 to 12 months, while approximately 1-in-20 patients require treatment for vasoconstriction, which typically occurs from 4 to 72 hours after the initial treatment.

Percutaneous transluminal coronary angioplasty (PTCA), also known as balloon angioplasty, is a treatment for coronary vessel stenosis. In typical PTCA procedures, a guiding catheter is percutaneously introduced into the cardiovascular system of a patient and advanced through the aorta until the distal end is in the ostium of the desired coronary artery. Using fluoroscopy, a guide wire is then advanced through the guiding catheter and across the site to be treated in the coronary artery. A balloon catheter is advanced over the guide wire to the treatment site. The balloon is then expanded to reopen the artery. The increasing popularity of the PTCA procedure is attributable to its relatively high success rate, and its minimal invasiveness compared with coronary by-pass surgery.

The benefit of balloon angioplasty, especially of the coronary arteries, has been amply demonstrated over the past decade. Angioplasty is effective to open occluded vessels that would, if left untreated, result in myocardial infarction or other cardiac disease or dysfunction. These benefits are diminished, however, by restenosis rates approaching 50% of the patient population that undergo the procedure. Restenosis is believed to be a natural healing reaction to the injury of the arterial wall that is caused by angioplasty procedures. The healing reaction begins with the clotting of blood at the site of the injury. The final result of the complex steps of the healing process is intimal hyperplasia, the migration and proliferation of medial smooth muscle cells (in a mechanism analogous to wound healing and scar tissue), until the artery is again stenotic or occluded. Such reocclusion may even exceed the clogging that prompted resort to the original angioplasty procedure. Accordingly, a huge number of patients experiencing a successful primary percutaneous transluminal coronary angioplasty (PTCA) procedure are destined to require a repeat procedure. The patient faces an impact on his or her tolerance and well being, as well as the considerable cost associated with repeat angioplasty.

To reduce the likelihood of reclosure of the vessel, it has become common practice for the physician to implant a stent in the patient at the site of the angioplasty or artherectomy procedure, immediately following that procedure, as a prophylactic measure. A stent is typically composed of a biologically compatible material (biomaterial) such as a biocompatible metal wire of tubular shape or metallic perforated tube. The stent should be of sufficient strength and rigidity to maintain its shape after deployment and to resist the elastic recoil of the artery that occurs after the vessel wall has been stretched. The deployment procedure involves advancing the stent on a balloon catheter to the designated site of the prior (or even contemporaneous) procedure under fluoroscopic observation. When the stent is positioned at the proper site, the balloon is inflated to expand the stent radially to a diameter at or slightly larger than the normal unobstructed inner diameter of the arterial wall, for permanent retention at the site. The stent implant procedure from the time of initial insertion to the time of retracting the balloon is relatively brief, and certainly far less invasive than coronary bypass surgery. In this fashion, the use of stents has constituted a beacon in avoidance of the complication, risks, potential myocardial infarction, need for emergency bypass operation, and repeat angioplasty that would be present without the stenting procedure.

Despite its considerable benefits, coronary stenting alone is not a panacea, as studies have shown that about 30% of the patient population subjected to that procedure will still experience restenosis (referred to hereinafter as “in-stent restenosis”). While this percentage is still quite favorable compared to the approximate 50% recurrence rate for patients who have had a PTCA procedure without stent insertion at the angioplasty site, improvement is nonetheless needed to reduce the incidence of in-stent restenosis. In the past few years, considerable research has been devoted worldwide to studying the mechanisms of in-stent restenosis. It has been shown that the very presence of the stent in the blood stream may induce a local or even systemic activation of the patient's hemostase coagulation system, resulting in local thrombus formation which, over time, may restrict the flow of blood.

To avoid this problem, various efforts have been undertaken to coat or treat the surface of the stent to prevent or minimize thrombus formation. One approach to reducing in-stent restenosis involves coating the stent with a biocompatible, non-foreign body-inducing, biodegradable polylactic acid of thin paint-like thickness in a range below 100 microns, and preferably about 10 microns thick. Animal research has shown that a 30% reduction in in-stent restenosis may be achieved using this technique. This thin coating on a metallic stent may be used to release drugs incorporated therein, such as hirudin and/or a platelet inhibitor such as prostacyclin (PGI.sub.2), a prostaglandin. Both of these drugs are effective to inhibit proliferation of smooth muscle cells, and decrease the activation of the intrinsic and extrinsic coagulation system. Therefore, the potential for a very significant reduction in restenosis has been demonstrated in these animal experiments.

Other coating techniques involve coating the stent with a biodegradable substance or composition which undergoes continuous degradation in the presence of body fluids such as blood, to self-cleanse the surface as well as to release thrombus inhibitors incorporated in the coating. Disintegration of the carrier occurs slowly through hydrolytic, enzymatic or other degenerative processes. The biodegradable coating acts to prevent the adhesion of thrombi to the biomaterial or the coating surface, especially as a result of the inhibitors in the coating, which undergo slow release with the controlled degradation of the carrier. Blood components such as albumin, adhesive proteins, and thrombocytes can adhere to the surface of the biomaterial, if at all, for only very limited time because of the continuous cleansing action along the entire surface that results from the ongoing biodegradation.

Materials used for the biodegradable coating and the slow, continuous release of drugs incorporated therein include synthetic and naturally occurring aliphatic and hydroxy polymers of lactic acid, glycolic acid, mixed polymers and blends. Alternative materials for those purposes include biodegradable synthetic polymers such as polyhydroxybutyrates, polyhydroxyvaleriates and blends, and polydioxanon, modified starch, gelatine, modified cellulose, caprolactaine polymers, acrylic acid and methacrylic acid and their derivatives. It is important that the coating have tight adhesion to the surface of the biomaterial, which can be accomplished by applying the aforementioned thin, paint-like coating of the biodegradable material that may have coagulation inhibitors blended therein, as by dipping or spraying, followed by drying, before implanting the coated biomaterial device.

Anti-proliferation substances may be incorporated into the coating carrier to slow proliferation of smooth muscle cells at the internal surface of the vascular wall. Such substances include corticoids and dexamethasone, which prevent local inflammation and further inducement of clotting by mediators of inflammation. Substances such as taxol, tamoxifen and other cytostatic drugs directly interfere with intimal and medial hyperplasia, to slow or prevent restenosis, especially when incorporated into the coating carrier for slow release during biodegradation. Local relaxation of a vessel can be achieved by inclusion of nitrogen monoxide (NO) or other drugs that release NO, such as organic nitrates or molsidomin, or SIN1, its biologically effective metabolite.

The amount and dosage of the drug or combination of drugs incorporated into and released from the biodegradable carrier material is adjusted to produce a local suppression of the thrombotic and restenotic processes, while allowing systemic clotting of the blood. The active period of the coated stent may be adjusted by varying the thickness of the coating, the specific type of biodegradable material selected for the carrier, and the specific time release of incorporated drugs or other substances selected to prevent thrombus formation or attachment, subsequent restenosis and inflammation of the vessel.

The biodegradable coating may also be applied to the stent in multiple layers, either to achieve a desired thickness of the overall coating or a portion thereof for prolonged action, or to employ a different beneficial substance or substances in each layer to provide a desired response during a particular period following implantation of the coated stent. For example, at the moment the stent is introduced into the vessel, thrombus formation will commence, so that a need exists for a top layer if not the entire layer of the coating to be most effective against this early thrombus formation, with a relatively rapid release of the incorporated, potent anticoagulation drug to complement the self-cleansing action of the disintegrating carrier. For the longer term of two weeks to three months after implantation, greater concern resides in the possibility of intimal hyperplasia that can again narrow or fully obstruct the lumen of the vessel. Hence, the same substance as was present or a different substance from that in the top layer might be selected for use in the application of the coating to meet such exigencies. Hirudin, for example, can be effective against both of these mechanisms or phenomena.

A still further technique for preventing restenosis involves the use of radiation. U.S. Pat. No. 4,768,507 to Fischell et al. proposes in the use of a special percutaneous insertion catheter for purposes of enhancing luminal dilatation, preventing arterial restenosis, and preventing vessel blockage resulting from intimal dissection following balloon and other methods of angioplasty. U.S. Pat. No. 4,779,641 and co-pending European patent application No. 92309580.6 disclose the use of an interbiliary duct stent, wherein radioactive coils of a wire are embedded into the interior wall of the bile duct to prevent restenotic processes from occurring. U.S. Pat. No. 4,448,691 and co-pending European patent application No. 90313433.6 disclose a helical wire stent provided for insertion into an artery following balloon angioplasty or atherectomy, which incorporates or is plated with a radioisotope to decrease the proliferation of smooth muscle cells. The disclosure teaches that the stent may be made radioactive by irradiation or by incorporating a radioisotope into the material of which the stent is composed. Another solution would be to locate the radioisotope at the core of the tubular stent or to plate the radioisotope onto the surface of the stent. The patent also teaches, aside from the provision of radioactivity of the stent, that an outer coating of anti-thrombogenic material might be applied to the stent.

U.S. Pat. No. 5,059,166 to Fischell et al. discloses a helical coil spring stent composed of a pure metal which is made radioactive by irradiation. Alternative embodiments disclosed in summary fashion in the patent include a steel helical stent which is alloyed with a metal that can be made radioactive, such as phosphorus (14.3 day half life); or a helical coil which has a radioisotope core and a spring material covering over the core; or a coil spring core plated with a radioisotope such as gold 198 (Au.sup.198, which has a half life of 2.7 days), which may be coated with an anti-thrombogenic layer of carbon.

Clinical basic science reports such as “Inhibition of neointimal proliferation with low dose irradiation from a beta particle emitting stent” by John Laird et al published in Circulation (93:529-536, 1996) describe creating a beta particle-emitting stent by bombarding the outside of a titanium wire with phosphorus. The implantation of phosphorus into the titanium wire was achieved by placing the P.sup.31 into a special vacuum apparatus, and then vaporizing, ionizing and, accelerating the ions with a higher voltage so that the P.sup.31 atoms become buried beneath the surface of the titanium wire in a thickness of about ⅓ micron. After exposing the wire together with the phosphorus radioisotope for several hours to a flux of slow neutrons part of the P.sup.31 atoms were converted into a P.sup.32, a pure beta particle emitter with a maximum energy of 1.709 megaelectron-volts, an average of 0.695 megaelectron-volts, and a half-life of 14.6 days.

Despite the convincing clinical results obtained by this method, practical application of the method in human patients raises considerable concerns. First, it is difficult to create a pure beta emitter from phosphorus if a stent is exposed to a flux of slow neutrons. In addition to converting phosphorus from P.sup.31 to P.sup.32, the metallic structure of the titanium wire will become radioactive. Therefore, about 20 days are needed to allow the radiation to decay, especially gamma radiation which originates from the titanium wire. Even worse is the situation where a metal such as stainless steel undergoes radioactive irradiation, resulting in production of unwanted .gamma. radiation and a wide range of short and long term radionuclei such as cobalt.sup.57, iron.sup.55, zinc.sup.65, molybdenum.sup.99, cobalt.sup.55. A pure beta radiation emitter with a penetration depth of about 3 millimeters is clearly superior for a radioactive stent for purposes of local action, side effects, and handling.

Reports have indicated that good results have been obtained with a radioactive wire inserted into the coronary arteries or into arteriosclerotic vessels of animals. Results obtained with a gamma radiation source from a wire stems from the deeper penetration of gamma radiation, which is about 10 mm. Assuming that the vessel is 3 to 4 mm in diameter, a distance of 2 to 4 mm depending on the actual placement of the wire toward a side wall has to be overcome before the radiation acts. Therefore, the clinical results that have been obtained with radioactive guide wires that have been inserted into the coronary arteries for a period ranging from about 4 to 20 minutes for delivery of a total dosage of about 8 to 18 Gray (Gy) have shown that gamma radiation has a beneficial effect while beta radiation from a wire is less favorable. On the other hand, gamma radiation which originates from a stainless steel stent such as composed of 316L is less favorable since the properties of .beta. radiation such as a short half-life and a short penetration depth are superior to .gamma. radiation originating from radioactive 316L with a long half-life and a deeper penetration since the proliferative processes of smooth muscle cell proliferation occur within the first 20 to 30 days and only in the very close vicinity of the stent.

In addition, a half-life which is too short such as one to two days considerably impacts on logistics if a metallic stent needs to be made radioactive. That is, by the tine the stent is ready for use, its radioactivity level may have decayed to a point which makes it unsuitable for the intended purpose.

Another technique for preventing in-stent restenosis involves providing stents seeded with endothelial cells (Dichek, D. A. et al Seeding of Intravascular Stents With Genetically Engineered Endothelial Cells; Circulation 1989; 80: 1347-1353). In that experiment, sheep endothelial cells that had undergone retrovirus-mediated gene transfer for either bacterial beta-galactosidase or human tissue-type plasmogen activator were seeded onto stainless steel stents and grown until the stents were covered. The cells were therefore able to be delivered to the vascular wall where they could provide therapeutic proteins. Other methods of providing therapeutic substances to the vascular wall by means of stents have also been proposed such as in international patent application WO 91112779 “Intraluminal Drug Eluting Prosthesis” and international patent application WO 90/13332 “Stent With Sustained Drug Delivery”. In those applications, it is suggested that antiplatelet agents, anticoagulant agents, antimicrobial agents, antimetabolic agents and other drugs could be supplied in stents to reduce the incidence of restenosis.

In the vascular graft art, it has been noted that fibrin can be used to produce a biocompatible surface. For example, in an article by Soldani et al., “Bioartificial Polymeric Materials Obtained from Blends of Synthetic Polymers with Fibrin and Collagen” International Journal of Artificial Organs, Vol. 14, No. 5, 1991, polyurethane is combined with fibrinogen and cross-linked with thrombin and then made into vascular grafts. In vivo tests of the vascular grafts reported in the article indicated that the fibrin facilitated tissue ingrowth and was rapidly degraded and reabsorbed. Also, in published European Patent Application 0366564 applied for by Terumo Kabushiki Kaisha, Tokyo, Japan, discloses a medical device such as an artificial blood vessel catheter or artificial internal organ is made from a polymerized protein such as fibrin. The fibrin is said to be highly nonthrombogenic and tissue compatible and promotes the uniform propagation of cells that regenerates the intima. Also, in an article by Gusti et al., “New Biolized Polymers for Cardiovascular Applications”, Life Support Systems, Vol. 3, Suppl. 1, 1986, “biolized” polymers were made by mixing synthetic polymers with fibrinogen and cross-linking them with thrombin to improve tissue ingrowth and neointima formation as the fibrin biodegrades. Also, in an article by Haverich et al., “Evaluation of Fibrin Seal in Animal Experiments”, Thoracic Cardiovascular Surgeon, Vol. 30, No. 4, pp. 215-22, 1982, the authors report the successful sealing of vascular grafts with fibrin. However, none of these teach that the problem of restenosis could be addressed by the use of fibrin and, in fact, conventional treatment with anticoagulant drugs following angioplasty procedures is undertaken because the formation of blood clots (which include fibrin) at the site of treatment is thought to be undesirable.

The present invention is directed at a method and apparatus for maintaining blood flow through vessels while preventing stenosis and restenosis in a fashion that overcomes, or at least reducing the effects of, one or more of the problems set forth above.

### SUMMARY OF THE INVENTION

The present invention solves the above-identified drawbacks with the prior art by providing a graft assembly which includes a length of graft conduit (autologous or synthetic) equipped with a first deployment assembly and a second deployment assembly at either end. The first deployment assembly includes an elastomeric sheath and a first stent ring. The second deployment assembly includes an elastomeric sheath and a second stent ring. The elastomeric sheaths may comprise any number of suitable materials having biocompatible and elastomeric characteristics. Each sheath is coupled or anchored to the exterior surface of one end of the graft conduit, while the remaining length of the sheaths are dimensioned to stretch and extend through the respective stent rings before being doubled back and coupled or anchored to the exterior of the stent rings.

The stent rings may start out in a contracted, crimped, or partially expanded state and may comprise any number of self-expanding and/or balloon-expandable types of stent structures known in the art. The variety of stent ring (self-expanding or balloon-expandable) dictates the type of delivery mechanism for transporting the graft assembly into a desired region within the patient for deployment. When using the balloon-expandable variety, it is preferred to crimp the stent rings in position on the balloon of a balloon catheter such that only the elastomeric sheaths are disposed between the stent rings and the balloon catheter. To do so, the elastomeric sheaths must be stretched and expanded from the anchor point on the exterior of the stent rings, past the end of the stent rings, and along the entire interior of the stent rings before extending towards the anchor point on the graft material. In this fashion, the graft conduit is maintained in between the stent rings during delivery into a patient, which minimizes the overall diameter of the graft assembly (for ease in placement) and protects the graft conduit from being damaged by the balloon during inflation.

In an important aspect of the present invention, the elastomeric sheaths are dimensioned such that, when stent rings are deployed (via self-expansion or balloon-expansion) within a blood vessel, the sheaths will contract in length and cause the ends of the graft conduit to be drawn generally equal to or past the outer ends of the stent rings and into a generally mating relationship with the inside of the blood vessel. This is once again due to the fact that, in preparation for delivery into a patient, the elastomeric sheaths must be stretched around the stent rings such that the graft material resides in between the stent rings during delivery. Upon deployment of the stent rings, the elastomeric sheaths will automatically contract in an effort to return to their natural, unstretched state and, in so doing, automatically pull the ends of the graft conduit into a mating relationship with the interior of the blood vessel. In this fashion, the extent to which blood flowing though the vessel will come into contact with the stent rings will be minimized if not eliminated altogether. This is a significant advantage in that permitting blood to contact or interface with non-autologous materials, such as stents, has been found to be a cause of the deposition of fatty materials, cellular debris, calcium, and/or blood clots that lead to stenosis and restenosis. By ensuring the blood interfaces only with autologous material, the development of stenosis and restenosis may be avoided or dramatically reduced.

### BRIEF DESCRIPTION OF THE DRAWINGS

Other objects and advantages of the invention will become apparent upon reading the following detailed description and upon reference to the drawings in which:

FIG. 1 is a graft assembly according to an illustrative embodiment of the present invention;

FIG. 2 is a cross-sectional view of the graft assembly of the present invention shown in FIG. 1;

FIG. 3 is a cross-sectional view illustrating a first step in the manufacture of the graft assembly shown in FIG. 1;

FIG. 3 is a cross-sectional view illustrating a first step in the manufacture of the graft assembly shown in FIG. 1;

FIG. 4 is a cross-sectional view illustrating a second step in the manufacture of the graft assembly shown in FIG. 1;

FIG. 5 is a cross-sectional view illustrating a third step in the manufacture of the graft assembly shown in FIG. 1;

FIG. 6 is a cross-sectional view illustrating a fourth step in the manufacture of the graft assembly shown in FIG. 1;

FIG. 7 is a cross-sectional view illustrating a fifth step in the manufacture of the graft assembly shown in FIG. 1;

FIG. 8 is a side view illustrating a graft assembly and a dual-balloon delivery catheter according to a first main embodiment of the present invention;

FIG. 9 is a partial sectional view illustrating the placement of the graft assembly of FIG. 8 in a partially occluded blood vessel via the dual-balloon delivery catheter,

FIG. 10 is a partial sectional view illustrating the deployment of the graft assembly of FIG. 8 in a partially occluded blood vessel via the dual-balloon delivery catheter,

FIG. 11 is a cross-sectional view illustrating the graft assembly of FIG. 8 in a fully deployed state within a partially occluded blood vessel;

FIG. 12 is a cross-sectional view illustrating the graft assembly of FIG. 8 in a fully deployed state within a stent deployed within a blood vessel;

FIG. 13 is a partial sectional view illustrating a graft assembly of a second main embodiment of the present invention being placed within a partially occluded blood vessel through the use of a single-balloon delivery catheter within a guide-catheter,

FIG. 14 is a partial sectional view illustrating a graft assembly of a third main embodiment of the present invention being placed within a partially occluded blood vessel through the use of a duck-bill delivery catheter within a guide-catheter,

FIG. 15 is a partial sectional view illustrating a graft assembly of a fourth main embodiment of the present invention being placed within a partially occluded blood vessel through the use of a delivery catheter having elongated holder rods within a guide-catheter;

FIG. 16 is a cross-sectional view of the graft assembly, delivery catheter, and guide catheter as taken through lines 16-16 in FIG. 15;

FIG. 17 is a partial sectional view illustrating a graft assembly of a fifth main embodiment of the present invention being placed within a partially occluded blood vessel through the use of a single-balloon delivery catheter;

FIG. 18 is a partial sectional view illustrating the deployment of the graft assembly of FIG. 17 in a partially occluded blood vessel via the single-balloon delivery catheter, and

FIG. 19 is a cross-sectional view illustrating the graft assembly of FIG. 17 in a fully deployed state within a partially occluded blood vessel.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

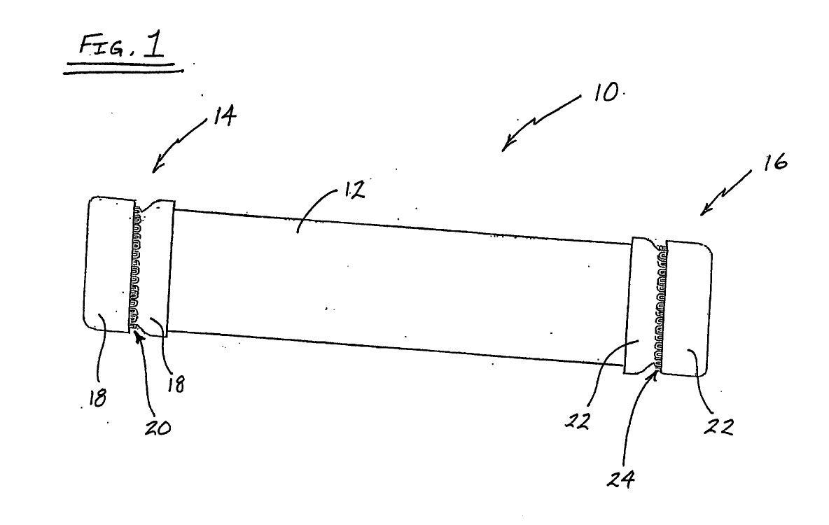

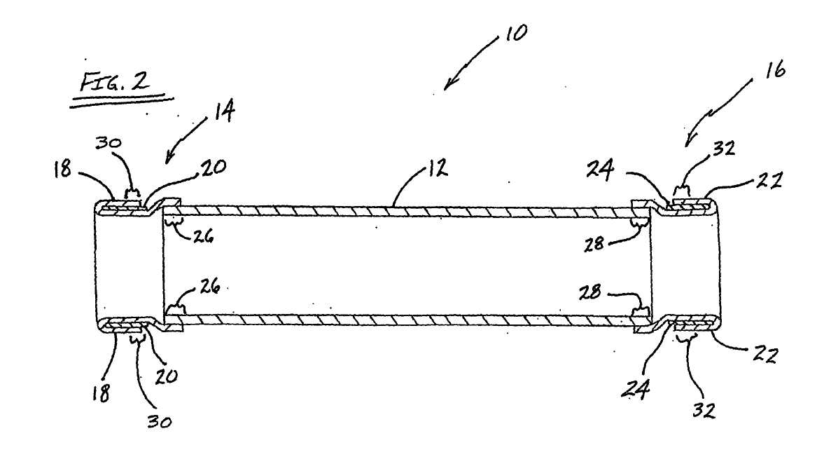

FIGS. 1 and 2 illustrate a graft assembly 10 according to one aspect of the present invention including a length of graft conduit 12 (autologous or synthetic) which; when deployed within a diseased or occluded vessel or duct, reestablishes sufficient flow therethrough and isolates the diseased or occluded region from the rest of the vessel by forming a lining along the interior surface of the diseased or occluded region. The graft conduit 12 is equipped with a first deployment assembly 14 and a second deployment assembly 16 at either end. The first deployment assembly 14 includes an elastomeric sheath 18 and a first anchor member 20. The second deployment assembly 16 includes an elastomeric sheath 22 and a second anchor member 24. As will be explained in greater detail below, each elastomeric sheath 18, 22 is coupled or anchored to the exterior surface of one end of the graft conduit 12, while the remaining length of the sheaths 18, 22 are dimensioned to extend stretch through the respective anchor members 20, 24 before being doubled back and coupled or anchored to the exterior of the anchor members 20, 24. Upon deployment, the graft assembly 10 reestablishes or maintains sufficient flow through diseased or occluded regions within a blood vessel. The graft assembly 10 also advantageously isolates the diseased or occluded region from the rest of the vessel by forming a lining (following deployment) along the interior surface of the diseased or occluded region.

Anchor members 20, 24 may comprise any number of self-deployable and/or balloon-deployable structures or devices, including but not limited to stents or stent-like devices known in the art (including self expanding and/or balloon-expandable stents). The anchor members 20, 24 may start out in the generally contracted state shown, or may be crimped or partially expanded depending upon the application, stent type and/or delivery mechanism. The type of anchor structure (i.e. self-expanding and/or balloon-expandable) dictates the type of delivery mechanism for transporting the graft assembly 10 into a desired region within the patient for deployment. As will be explained in greater detail below, when using the balloon-deployable variety, it is preferred to crimp the anchor members 20, 24 in position on the balloon of a balloon catheter such that only the elastomeric sheaths 18, 22 are disposed between the anchor members 20, 24 and the balloon catheter. To do so, the elastomeric sheaths 18, 22 must be stretched and expanded from the anchor point on the exterior of the anchor elements 20, 24, past the end of the anchor elements 20, 24, and along the entire interior of the anchor members 20, 24 before extending towards the anchor point on the graft conduit 12. In this fashion, the graft conduit 12 is maintained in between the anchor members 20, 24 during delivery into a patient, which minimizes the overall diameter of the graft assembly 10 (for ease in placement) and protects the graft conduit 12 from being damaged by the balloon during inflation.

In an important aspect of the present invention, the elastomeric sheaths 18, 22 are dimensioned such that, when anchor members 20, 24 are deployed (via self-expansion or balloon-expansion) within a blood vessel, the sheaths 18, 22 will contract in length and cause the ends of the graft conduit 12 to be drawn generally equal to or past the outer ends of the anchor members 20, 24 and into a generally mating relationship with the inside of the blood vessel. This is once again due to the fact that, in preparation for delivery into a patient, the elastomeric sheaths 18, 22 must be stretched around the anchor members 20, 24 such that the graft conduit 12 resides in between the anchor members 20, 24 during delivery. Upon deployment of the anchor members 20, 24, the elastomeric sheaths 18, 22 will automatically contract in an effort to return to their natural, unstretched state and, in so doing, automatically pull the ends of the graft conduit 12 into a mating relationship with the interior of the blood vessel. In this fashion, the extent to which blood flowing though the vessel will come into contact with the anchor members 20, 24 will be minimized if not eliminated altogether. This is a significant advantage in that permitting blood to contact or interface with non-autologous materials, such as stents, has been found to be a cause of the deposition of fatty materials, cellular debris, calcium, and/or blood clots that lead to stenosis and restenosis. By ensuring the blood interfaces only with the graft material, the development of stenosis and restenosis may be avoided or dramatically reduced. The graft assembly 10 reestablishes sufficient flow through diseased or occluded regions and serves to isolate these diseased or occluded regions from the rest of the vessel by forming a lining along the interior surface of the diseased or occluded region.

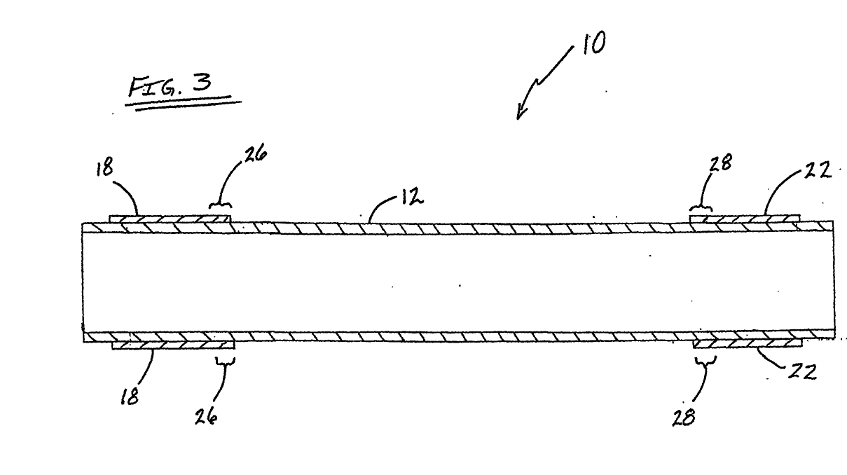

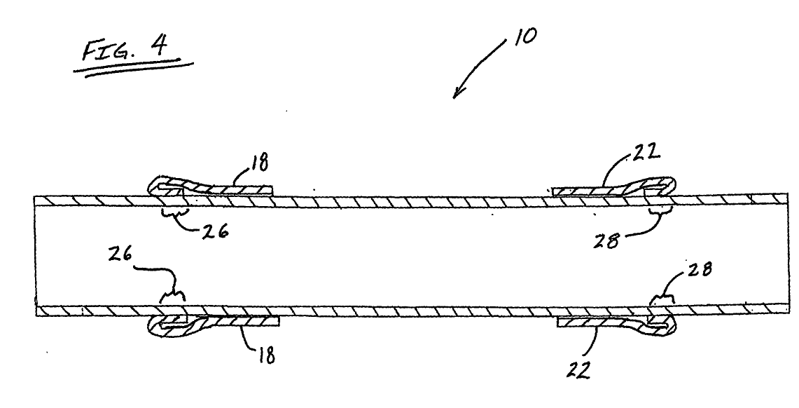

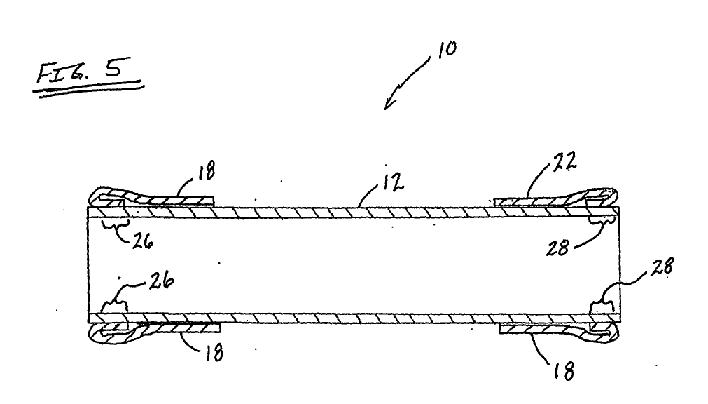

The design of the graft assembly 10 also facilitates ease of manufacture. Referring to FIG. 3, the first step in manufacturing the graft assembly 10 involves fixedly coupling the inner ends of the elastomeric sheaths 18, 22 to the graft conduit 12, such as in the regions shown generally at 26, 28. The task of fixedly coupling the sheaths 18, 22 to the graft conduit 12 may be performed using any number of suitable techniques, devices, or compositions. These may include, but are not necessarily limited to, the use of sutures, staples, adhesives, fusion, or any other manner of establishing a fixed relationship between the ends of the sheaths 18, 22 and the graft conduit 12. Other than at the regions shown generally at 26, 28, the remainder of the elastomeric sheaths 18, 22 remain disposed along at least a portion of the exterior of the graft conduit 12 but are in no way fixed to the graft conduit 12. This facilitates performing the next step in manufacturing the graft assembly 10 of the present invention, shown in FIG. 4, which involves folding the elastomeric sheaths 18, 22 inwardly past the coupling regions 26, 28. In this fashion, the ends of the graft conduit 12 are left exposed, enabling the next manufacturing step shown in FIG. 5—trimming the exposed ends of the graft conduit 12. A benefit of this “coupling-folding-trimming” arrangement is that it allows a person preparing the graft assembly 10 (such as a surgeon or medical assistant) to tailor the length of the graft conduit 12 to suit a particular application, as well as providing the ability to establish the coupling regions 26, 28 at or near the ends of the graft conduit 12. As will be explained in greater detail below, this latter point is important in that it facilitates positioning the ends of the graft conduit 12 in a generally mating relationship with the interior of the vessel wall such that little, if any, blood can contact anything other than the graft conduit 12, thereby preventing or reducing the onset of stenosis or restenosis.

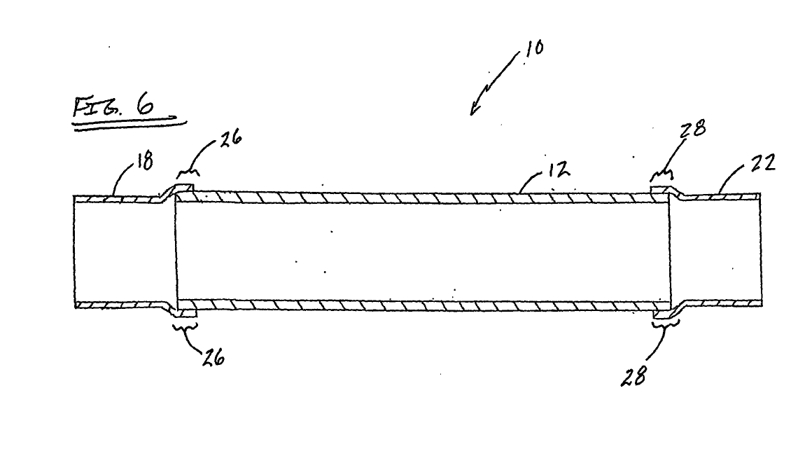

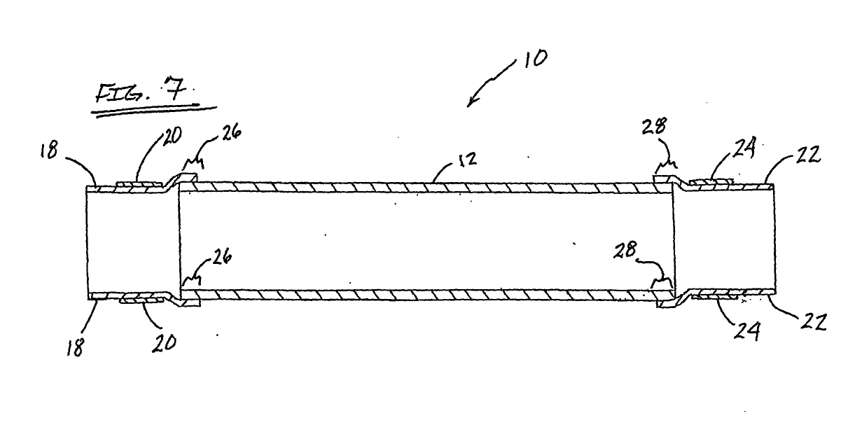

With the ends of the graft conduit 12 trained, the elastomeric sheaths 18, 22 may then be unfolded as in FIG. 6. At this point, the anchor members 20, 24 must be coupled or anchored to the elastomeric sheaths 18, 22. One way of accomplishing this is to position the anchor members 20, 24 over the respective elastomeric sheaths 18, 22 (as shown in FIG. 7) and then stretch and fold the elastomeric sheaths 18, 22 back over the exterior of the anchor members 20, 24 for attachment to the anchor members 20, 24 (as shown in FIG. 2). This attachment or anchoring, represented generally at 30, 32 in FIG. 2, may be performed using any number of suitable techniques, devices, or compositions, including but not limited to sutures, staples, adhesives, fusion, or any other manner of establishing a fixed relationship between the outer ends of the sheaths 18, 22 and the anchor members 20, 24. The elastomeric nature of the sheaths 18, 22 is an important feature of the present invention in that it allows the sheaths 18, 22 to stretch and expand from the anchor point on the exterior of the anchor members 20, 24, past the end of the anchor members 20, 24, and along the entire interior of the anchor members 20, 24 before extending towards the anchor point on the graft conduit 12. The graft conduit 12 may thus be maintained in between the anchor members 20, 24 during delivery into a patient. This has the two-fold benefit of minimizing the overall diameter of the graft assembly 10 (facilitating placement) and protecting the graft conduit 12 from being damaged during deployment, such as via balloon inflation when using balloon-expandable stent rings.

The various components forming the graft assembly 10 of the present invention may be formed of any number of suitable materials and dimensioned in any number of different fashions depending upon the application. The following recitations are set forth by way of example only. The graft conduit 12 may be comprised of a length of autologous blood vessel harvested from (or grown in a lab based on the DNA of) the very patient having the graft assembly 10 deployed. The graft conduit 12 may also comprise any number of synthetic materials (now existing or later-developed) exhibiting similar characteristics as autologous grafts. The graft conduit 12 may be dimensioned having a length in the range of between 5 and 50 mm (20 mm being preferred), a diameter in the range of between 2 and 5 mm (2 mm being preferred), and a wall thickness in the range of between 0.01 and 0.5 mm (0.375 mm being preferred). The anchor members 20, 24 may be comprised of any material suitable to provide structural support within the blood vessel following deployment, including but not limited to stainless steel, biocompatible composites and/or Nitonol. Anchor members 20, 24 may be dimensioned having a length in the range of between 0.5 mm and 50 mm (2.5 mm being preferred), a collapsed diameter in the range of between 1 mm and 3 mm (1.6 mm being preferred), an expanded diameter in the range of between 2 mm and 5 mm (3 mm being preferred), and a wall thickness (while expanded) in the range of between 0.02 mm and 0.2 mm (0.15 being preferred). The elastomeric sheaths 18, 22 may be comprised of any number of elastomeric materials, including but not limited to silicone or any other polymers or compositions having contractility characteristics. The elastomeric sheaths 18, 22 may be dimensioned in any range of length, diameter, and wall thickness suitable to permit the necessary stretching between the anchor members 20, 24 and the graft conduit 12 and subsequent contraction to bias the ends of the graft conduit 12 to a point equal to or past the ends of the anchor members 20, 24 following the deployment of the anchor members 20, 24. In this regard, it should be noted that the elastomeric sheaths 18, 22 shown throughout the drawings are depicted (in the interest of clarity) having a width substantially greater than would actually be found in practice. For example, the width of the elastomeric sheaths 18, 22 may range from 0.05 mm (stretched) and 0.15 mm (unstretched).

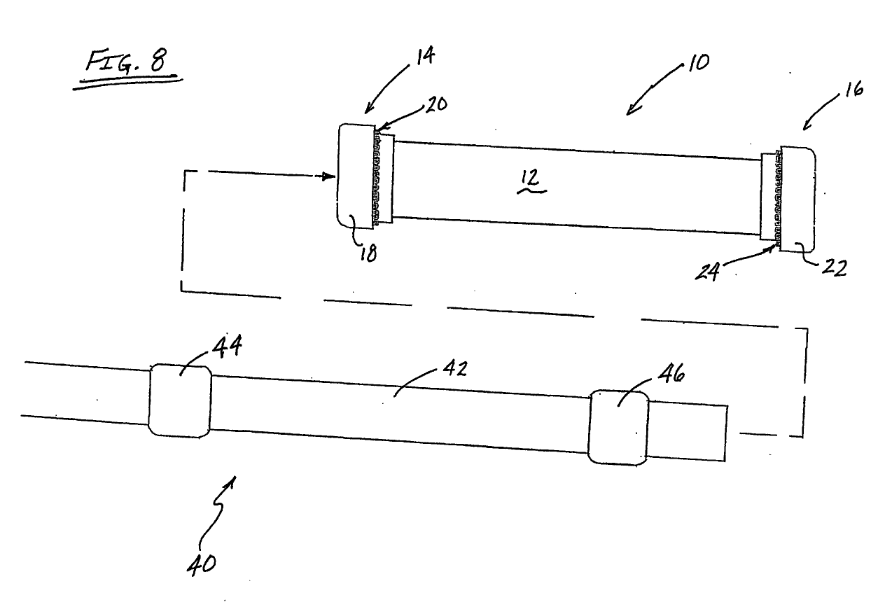

A first main embodiment of the graft assembly 10 of the present invention will now be described with reference to FIGS. 8-12. According to this embodiment, the first and second deployment assemblies 14, 16 are equipped with balloon-expandable anchor members 20, 24. As used herein, the term “balloon-expandable” is meant to include any type of stent or scaffolding type structure for placement within a blood vessel which can be expanded through the use of a balloon or any other mechanism including but not limited to those employing mechanical, hydraulic, and/or pneumatic techniques for expanding the anchor members 20, 24. One such device is a dual-balloon delivery catheter 40 of the type shown in FIG. 8. The dual-balloon delivery catheter 40 includes a catheter body 42 having a first balloon 44 and a second balloon 46. The first and second balloons 42, 44 are selectively inflatable, such as through the use of one or more fluid sources (not shown) communicatively coupled to the balloons 42, 44 through one or more lumens (not shown) disposed within the wall of the catheter body 42.

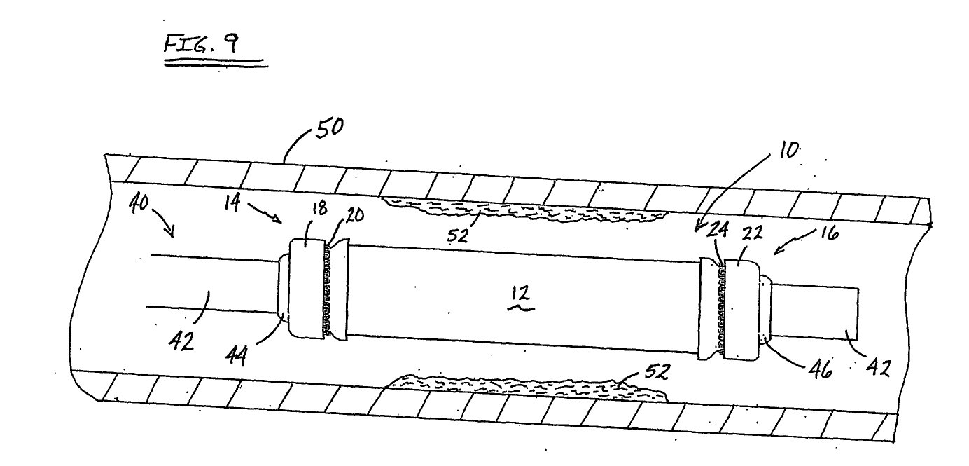

As shown in FIG. 9, the dual-balloon delivery catheter 40 is dimensioned to carry or deliver the graft assembly 10 of the present invention into a selected region within a blood vessel 50 for deployment. In the embodiment shown, the selected region is one including a flow restriction 52 (such as due to the build-up or deposit of fatty materials, cellular debris, calcium, and/or blood clots) capable of causing stenosis. The dual-balloon delivery catheter 40 may be selectively positioned such that the first and second deployment assemblies 14, 16 are disposed on either side of the flow restriction 52. This may be facilitated through the use of a guide-wire (not shown) that is first introduced into the patient's vasculature and advanced to the desired region through the use of traditional guidance techniques, including but not limited to flouroscopy, after which point the delivery catheter 40 (carrying the graft assembly 10) may be advanced over the guide-wire to the desired position. The dual-balloon delivery catheter 40 may be dimensioned in any number of suitable fashions, such as by providing the catheter body 42 having a diameter in the range of between 5 and 9 French and a length capable of reaching, for example, a coronary artery from an incision point in the femoral artery.

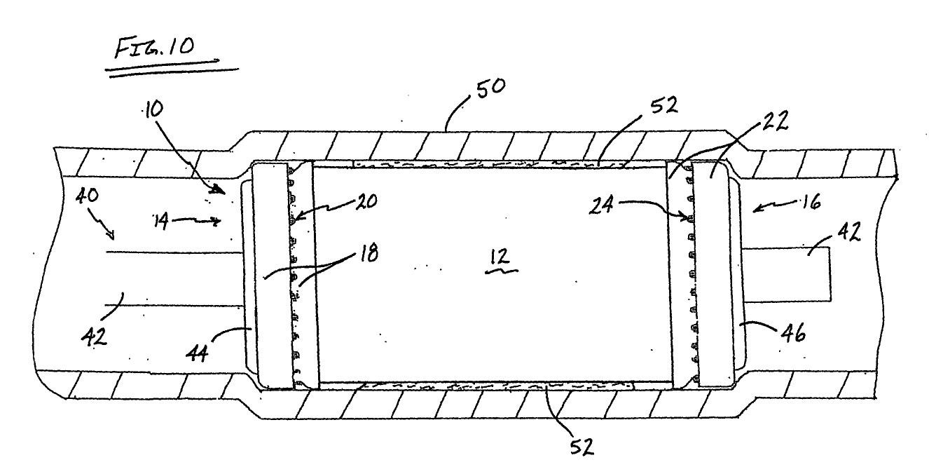

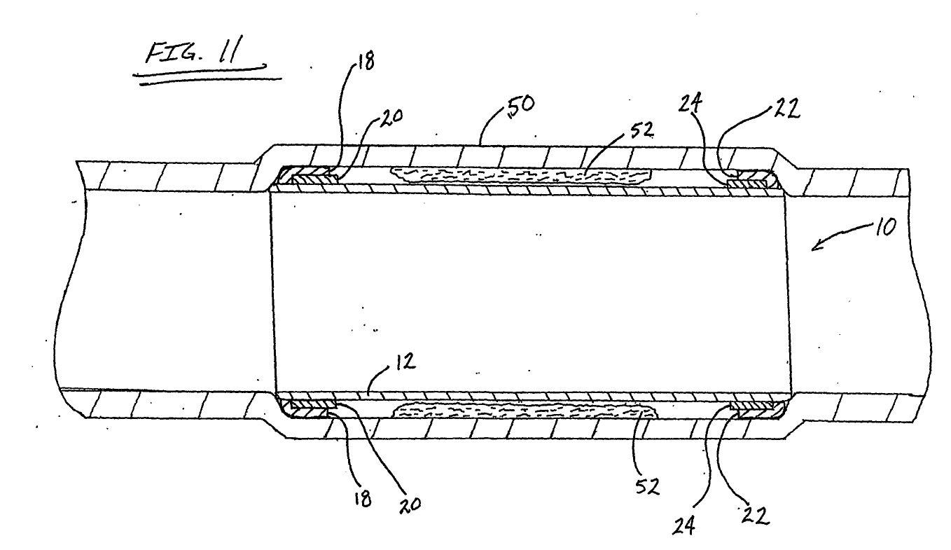

After the graft assembly 10 of the present invention has been positioned in the selected region, the first and second balloons 44, 46 may be inflated (simultaneously or in series) as shown in FIG. 10. As can be seen, the inflation of the balloons 44, 46 causes the anchor members 20, 24, respectively, to expand to a greatly increased diameter, preferably to the point where the vessel wall 50 distends in the region between the anchor members 20, 24. As will be explained in greater detail below, this distention is advantageous in that it creates space within the vessel 50 within which to position the graft assembly 10 of the present invention. After deployment, the balloons 44, 46 may be deflated and the catheter body 42 removed from the patient. As shown in FIG. 11, the elastomeric sheaths 18, 22 are dimensioned to contract upon the removal of the delivery catheter 40. In this embodiment, this contraction is sufficient to retract the elastomeric sheaths 18, 22 into the space created by the distention of the blood vessel 50. This has the advantageous effect of pulling the ends of the graft conduit 12 into a generally mating relationship with the interior of the blood vessel 50. This, in turn, advantageously prohibits blood flowing within the vessel 50 from contacting the anchor members 20, 24. In this fashion, the deposit of new flow restrictions 52 is prevented and stenosis thwarted.

The elastomeric sheaths 18, 22 are shown and described throughout as biasing the ends of the graft conduit 12 to a point equal to or past the ends of the anchor members 20, 24 and into a generally mating relationship with the interior of the vessel 50. However, it should be noted that, in certain applications and instances, it may be acceptable to dimension the elastomeric sheaths 18, 22 such that ends of the graft conduit 12 do not extent equal to or past the ends of the anchor members 20, 24. For example, it may be acceptable for the sheaths 18, 22 to remain extended (fully or partially) along the interior of the anchor members 20, 24 after deployment, such as where it is found that the blood-sheath interface is non-thrombogenic or unlikely to cause the formation of flow restrictions. It is similarly anticipated that the elastomeric sheaths 18, 22 be dimensioned so as to wrap the ends of the graft conduit 12 past the ends of the anchor members 20, 24 and into the spaced formed by the distention of the vessel 50. That is to say, the ends of the graft conduit 12 maybe wrapped so far as to extend at least partially along the exterior surface of the anchor members 20, 24. The common denominator between all these embodiments is that the blood flow is prevented from contacting the anchor members 20, 24, thereby preventing the formation of flow restrictions thereon.

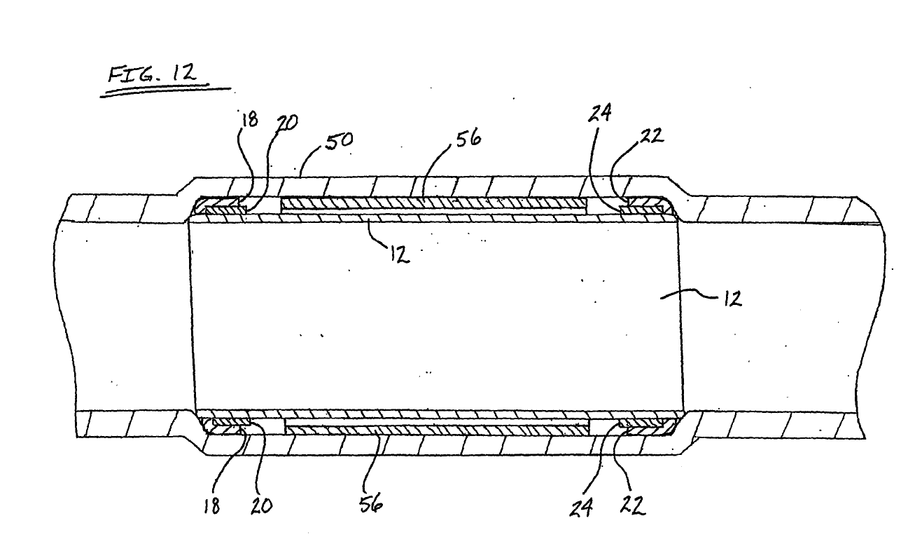

The principles of the present invention also have great applicability in both the prevention and elimination of in-stent restenosis. In-stent restenosis occurs when a stent that has been previously deployed in a patient undergoes a subsequent build-up of flow restrictions to the point that additional procedures may be required to restore sufficient blood flow therethrough. This feature of preventing and eliminating in-stent restenosis is evident with reference to FIG. 12.

The proactive step of preventing in-stent restenosis may be accomplished by deploying the graft assembly 10 of the present invention immediately following the deployment of a stent 56 within a selected region within the blood vessel 50. In this fashion, patients who undergo a stent placement procedure (such as to overcome an initial bout of stenosis in the blood vessel 50) will be less likely to suffer from in-stent restenosis due to the fact that the stent 56 will be lined, thereby preventing blood from contacting the stent 56. This, once again, is based on the advantageous feature of having the elastomeric sheaths 18, 22 bias the ends of the graft conduit 12 into a generally mating relationship with the interior of the vessel wall 50 at points equal to or past the anchor members 20, 24. The placement of the stent 56 is also advantageous in that prevents collapse the vessel wall 50 into the graft conduit 12 following deployment. In this fashion, the method of deploying the graft assembly 10 of the present invention into a stent 56 that has been intentionally and recently deployed in a blood vessel represents a significant advantage in terms of the prevention of restenosis and ensuing for unimpeded blood flow.

The reactive step of eliminating or treating in-stent restenosis may be accomplished by deploying the graft assembly 10 of the present invention after restenosis has developed within the stent 56. The graft assembly 10 of the present invention may be employed in combination with current techniques for treating in-stent restenosis. For example, the graft assembly 10 may be deployed following the use of miniaturized rotary devices designed to break up and remove some or all of the stenotic build-up within the stent 56. In this fashion, the end result will be similar or identical to that shown in FIG. 12, particularly if the stent 56 is expanded past its original deployment diameter before deployment of the graft assembly 10 (via a balloon catheter) or after deployment of the graft assembly 10 (via self-expansion). In either case, the stent 56 is lined along its interior surface by the deployed graft conduit 12, thereby eliminating the restenosis within the stent 56. Although not shown, it is to be readily appreciated that the graft assembly 10 maybe deployed within the stent 56 without removing the stenotic material disposed therein. In that case, stenotic material would be sandwiched between the exterior surface of the graft conduit 12 and the interior surface of the stent 56. This would advantageously prevent the continued narrowing of the lumen of the stent 56 and thereby maintain blood flow at adequate levels. With in-stent restenosis rates approaching 50% of the patient population that undergo PTCA procedures, the ability of the graft assembly 10 of the present invention to eliminate and prevent in-stent restenosis represents a major advancement in interventional medicine.

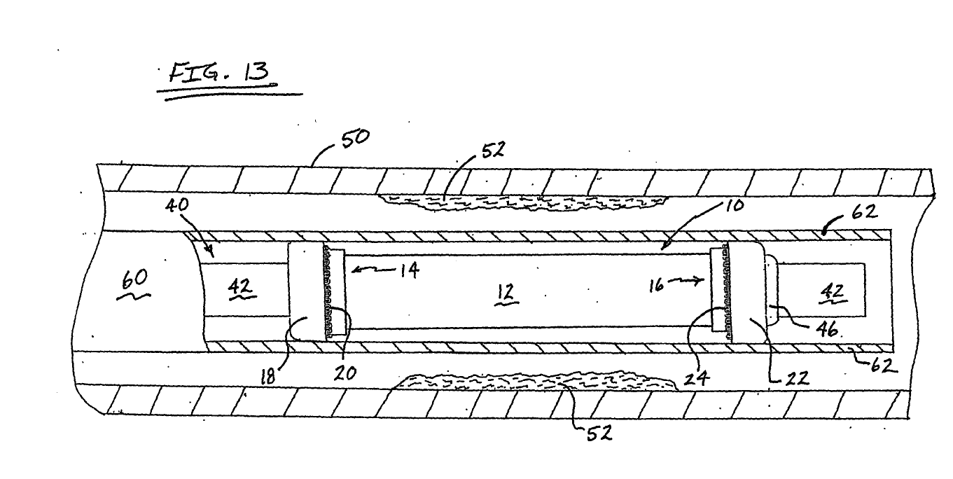

A second main embodiment of the graft assembly 10 of the present invention will now be described with reference to FIG. 13. According to this embodiment, anchor member 20 of the first deployment assembly 14 is self-expanding, while anchor member 24 of the second deployment assembly 16 is balloon-expandable. As used herein, the term “self-expanding” is meant to include any stent or scaffolding structure for placement within a blood vessel capable of expanding, generally speaking by itself and without the aid of additional mechanical devices. Based on the single balloon-expandable anchor member 24, the delivery catheter 40 need only comprise a single-balloon catheter having the distal balloon 46 coupled to the delivery catheter body 42 and operable in the same manner described above. The self-expanding nature of anchor member 20 requires a restraint mechanism to prevent the anchor member 20 from expanding until properly placed within the blood vessel 50. In the embodiment shown, this is accomplished through the use of a guide catheter 60 dimensioned to receive the graft assembly 10 and delivery catheter 40. Guide catheter 60 is shown in partial cross-section to illustrate the manner in which the wall 62 thereof cooperates to enclose and thereby restrain anchor member 20 of the first deployment assembly 14.

In use, a guide wire (not shown) may first be advanced into the desired location using traditional interventional cardiology guidance techniques. At this point, the guide catheter 60 may be advanced along the guide-wire by itself or with the delivery catheter 40 (carrying the graft assembly 10) disposed therein. In order for the guide catheter 60 to be advanced along the guide-wire by itself, the delivery catheter 40 must be capable of sliding through the inner lumen of the guide catheter 60 thereafter without disrupting the configuration of the first deployment assembly 14 or damaging any portion of the graft assembly 10. In either event, the end result is the placement of the guide catheter 60 in the desired location within the vessel 50, with delivery catheter 40 disposed within the guide catheter 60 as shown in FIG. 13. To deploy the graft assembly 10, the guide catheter 60 is first withdrawn past the second deployment assembly 16 such that the balloon 46 can be inflated to deploy anchor member 24. The delivery catheter 40 may then be withdrawn from the gaide catheter 60, after which point the guide catheter 60 is withdrawn to allow the self-expansion of anchor member 20 of the first deployment assembly 14. Although not shown, the graft assembly 10 thus resides within the blood vessel 50 in generally the same fashion as in the fully deployed state shown and described above with reference to FIGS. 11 and 12. It will also be appreciated that, although the entire delivery catheter 40 is shown disposed within the guide catheter 60 in FIG. 13, it is only necessary that the self-expanding first deployment assembly 14 be disposed therein in order to restrain the first anchor member 20.

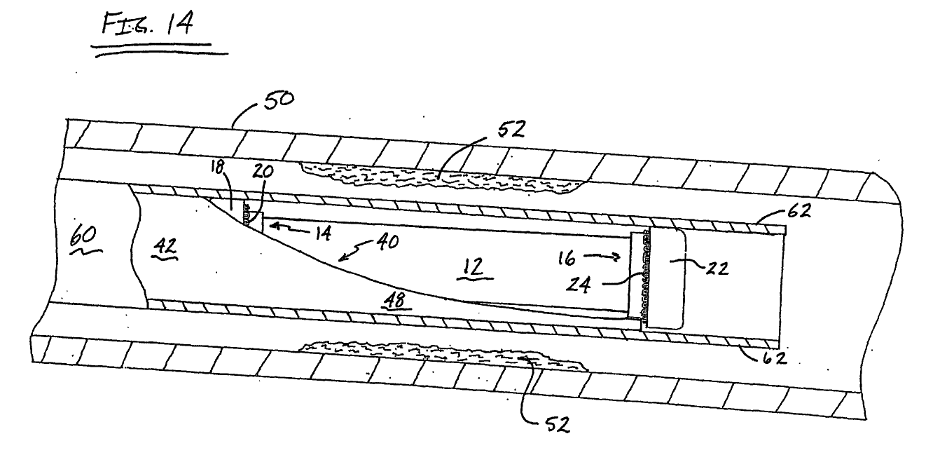

FIG. 14 illustrates a third main embodiment of a graft assembly 10 of the present invention. According to this embodiment, anchor members 20, 24 are both self-expanding and, consequently, require a restraint mechanism to prevent expansion until properly placed within the blood vessel 50. In the embodiment shown, this is accomplished through the combined use of a guide catheter 60 and a modified delivery catheter 40. As above, the guide catheter 60 is dimensioned to receive the graft assembly 10 and delivery catheter 40. The delivery catheter 40, however, includes a catheter body 42 terminating with a duck-bill portion 48. Duck-bill portion 48 has a generally tapered opening which is dimensioned to receive the first deployment assembly 14 at its proximal end and to abut a portion of the second deployment assembly 16 at its distal end. Once the guide catheter 60 and delivery catheter 40 are positioned as shown in FIG. 14, the guide catheter 60 maybe withdrawn over the delivery catheter 40. The distal end of the duck-bill portion 48 serves to maintain the second deployment assembly 16 in position while guide catheter 60 is being withdrawn. Once exposed, anchor member 24 of the second deployment assembly 16 will deploy. The first deployment assembly 14 resides within the proximal end of the duck-bill portion 48 until the guide catheter 60 is withdrawn therefrom, at which point the anchor member 20 will deploy. At that point, the graft assembly 10 resides within the blood vessel 50 in generally the same fashion as in the fully deployed state shown and described above with reference to FIGS. 1I and 12.

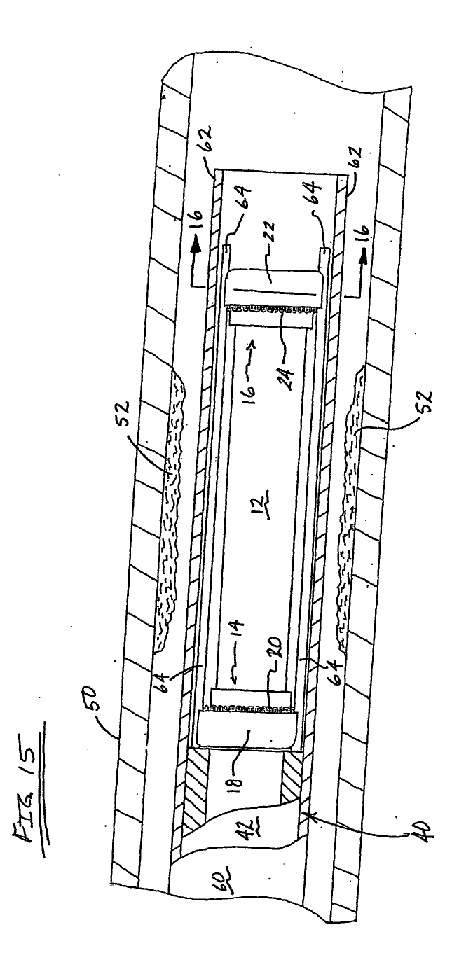

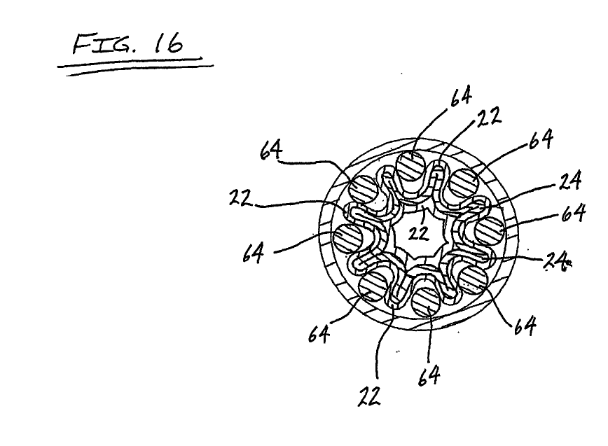

FIGS. 15 and 16 illustrate a graft assembly 10 of a fourth main embodiment of the present invention. The anchor members 20, 24 are self-expanding as in the embodiment shown and described above with reference to FIG. 14. The restraint mechanism, while it employs a guide catheter 60 of the type described above, involves yet another type of delivery catheter 40. The guide catheter 60 is dimensioned to receive the graft assembly 10 and delivery catheter 40. The delivery catheter 40 includes a catheter body 42 having a plurality of elongated rods 64 extending from a distal end thereof. As shown in FIG. 16, the rods 64 cooperate within a plurality of undulations formed by the anchor members 20, 24 to maintain the graft assembly 10 in the proper position within the blood vessel 50 as the guide catheter 60 is withdrawn. Once the guide catheter 60 is withdrawn, the elongated rods 64 maybe retracted into lumens formed within the wall of the catheter body 42 and, in so doing, release the second then first deployment assemblies 16, 14, respectively. In an alternate embodiment, the elongated rods 64 may be fixed in position but constructed in such a manner that they either self-expand (such as via Nitonol) or are pliable or controllable enough to allow the first and second deployment assemblies 14, 16 to deploy the graft conduit 12 according to the present invention. For example, the elongated rods 64 may permit the second deployment assembly 16 to first deploy and thereby provide enough purchase (that is, gripping or retaining ability) against the vessel wall 50 such that the delivery catheter 40 could be withdrawn until the first deployment assembly 14 deploys. Once deployed, the graft assembly 10 resides within the blood vessel 50 in generally the same fashion as in the fully deployed state shown and described above with reference to FIGS. 11 and 12.

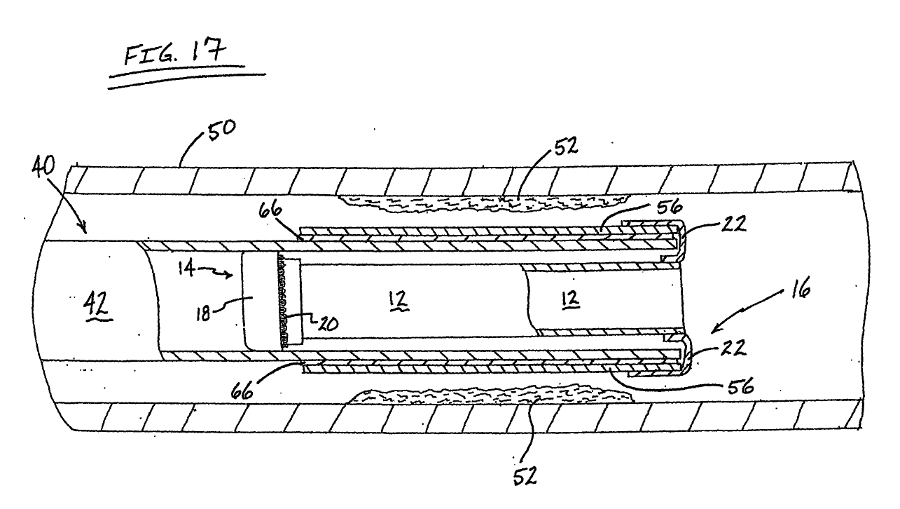

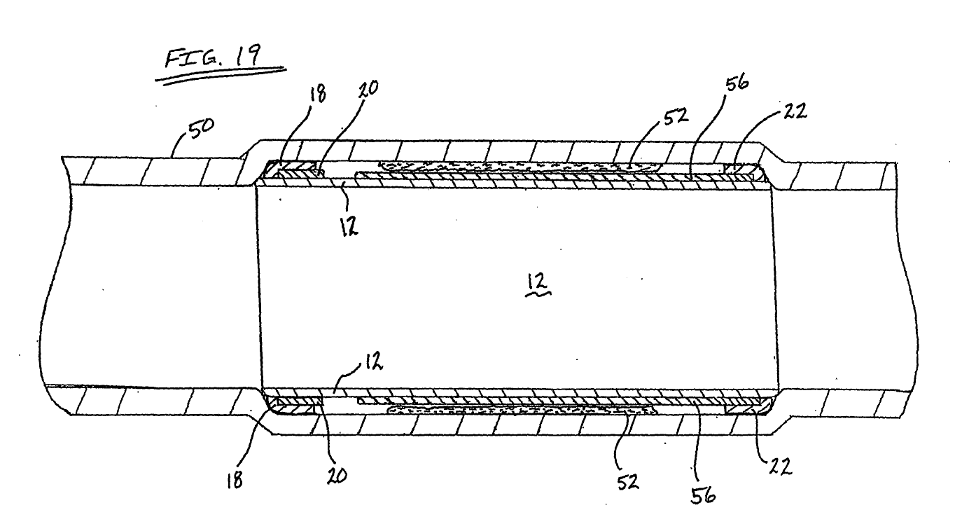

FIGS. 17-19 illustrate a graft assembly 10 of a fifth main embodiment of the present invention. According to this embodiment, the anchor member 20 of the first deployment assembly 14 is self-expanding. The second deployment assembly 16 is balloon-expandable, although it employs a full stent 56 as opposed to the anchor member 24 disclosed above. In this embodiment, the restraint mechanism for the first anchor member 20 comprises a modified delivery catheter 40 having an internal lumen for receiving the first deployment assembly 14. The delivery catheter 40 is also equipped with a selectively inflatable balloon 66 for deploying the stent 56 of the second deployment assembly 16. Once the delivery catheter 40 is positioned in the desired region within the blood vessel 50 (i.e. via a guide-wire), the balloon 66 is inflated as shown in FIG. 18. As can be seen, this serves to distend the vessel wall 50 according to the “space creation” aspects of the present invention. Once stent 56 is fully expanded, the balloon 66 may be deflated and the delivery catheter 40 withdrawn util the first deployment assembly 14 is released from within the lumen of the catheter body 42, producing the fully deployed graft assembly 10 shown in FIG. 19. The purchase created between the stent 56 and the vessel wall 50 is sufficient to overcome any drag between the elastomeric sheath 18 of the first deployment assembly 14 and the catheter body 42 as the delivery catheter 40 is being withdrawn. In so doing, the graft assembly 10 is quite easy to deploy.

Another benefit of this embodiment is that the use of the stent 56 allows a physician to deploy the graft assembly 10 with little or no preparation of the flow restriction materials 52 within the blood vessel 50. That is to say, the increased rigidity and expandability of the stent 56 provides the ability to position the graft assembly 10 in the desired location over an occluded region and simply deploy the stent 56 via the balloon 66 to distend the vessel wall 50. This is advantageous in that it also serves to move the stenotic material 52 out of the original flow path of the vessel 50, thereby obviating (or at least reducing) the need to perform preparatory procedures to remove such stenotic material 52 before deployment. This may translate into substantially reduced time required to perform such deployment procedures, with an attendant reduction in associated costs. This arrangement is also advantageous in terms of the space created by this distention of the vessel wall 50. More specifically, the space created by the deployment of the anchor member 20 and stent 56 is sufficient to accommodate the elastomeric sheaths 18, 22, respectively, as they contract to bias the ends of the graft conduit 12 into a generally mating relationship with the interior of the vessel 50.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the invention is to cover all modifications, equivalents, and alternative falling within the spirit and scope of the invention as defined by the appended claims.

1. An apparatus for restoring or maintaining flow through a vessel or duct within a living body, comprising:
a graft conduit having a first and second ends and an interior lumen extending therebetween, and
first and second anchor members coupled to said first and second ends of said graft conduit, said anchor members capable of being deployed within said vessel or duct to establish a flow path through said interior lumen of said graft.

2. The apparatus of claim 1 and further, wherein said first and second anchor members are coupled to said first and second ends of said graft conduit with first and second coupling elements.

3. The apparatus of claim 2, wherein the first and second coupling elements are elastomeric.

4. The apparatus of claim 3, wherein the first and second coupling elements serve to bias the first and second ends of the graft conduit substantially into abutment against the interior lumen of the vessel or duct.

5. The apparatus of claim 1, wherein the graft conduit isolates a portion of the interior lumen of the vessel or duct from the flow of blood therethrough.

6. The apparatus of claim 1, wherein at least one of the first and second anchor members is deployable via self-expansion or forced-expansion.

7. The apparatus of claim 1, wherein said first and second anchor members are deployable via forced-expansion, and wherein said graft conduit and said first and second anchor members are delivered into the vessel or duct via a deployment assembly having at least two expansion members.

8. The apparatus of claim 7, wherein the deployment assembly comprises a delivery catheter having first and second balloons capable of being inflated to deploy the first and second anchor members within the vessel or duct.

9. The apparatus of claim 1, wherein the graft conduit comprises a length of bio-compatible synthetic conduit.

10. The apparatus of claim 1, wherein the graft conduit comprises a length of autologous blood vessel obtained by one of harvesting said autologous blood vessel from the living body and growing said autologous blood vessel using bio-engineering techniques.

11. The apparatus of claim 1, wherein the graft conduit is dimensioned having a length ranging from 5 mm to 50 mm.

12. The apparatus of claim 1, wherein the graft conduit is dimensioned having a diameter ranging from 2 mm to 5 mm.

13. The apparatus of claim 1, wherein the graft conduit is dimensioned having a wall thickness ranging from 0.01 mm to 0.5 mm.

14. The apparatus of claim 1, wherein the anchor members are constructed to provide sufficient rigidity to maintain the first and second ends of said graft conduit open upon deployment within said vessel or duct.

15. The apparatus of claim 1, wherein the anchor members are constructed from at least one of stainless steel a biocompatible composite, and Nitonol.

16. The apparatus of claim 1, wherein the anchor members are dimensioned having a length ranging from 0.5 mm to 50 mm.

17. The apparatus of claim 1, wherein the anchor members are dimensioned having an initial diameter ranging from 1 mm to 3 mm.

18. The apparatus of claim 1, wherein the anchor members are dimensioned having a diameter upon deployment ranging from 2 mm to 5 mm.

19. The apparatus of claim 1, wherein the anchor members are dimensioned having a wall thickness ranging from 0.02 mm to 0.2 mm.

20. The apparatus of claim 3, wherein the coupling elements are constructed from at least one of silicone and any polymer or composition having contractility characteristics.

21. The apparatus of claim 3, wherein the coupling elements are dimensioned having a width when deployed of approximately 0.05 mm and a width prior to deployment of approximately 0.15 mm.

22. The apparatus of claim 1, wherein the anchor members are deployed via self-expansion and are contained within a first tubular element during introduction into the vessel or duct.

23. The apparatus of claim 22, wherein the anchor members are deployed through the use of a second tubular element disposed within said first tubular element.

24. The apparatus of claim 23, wherein the second tubular element has a duck-bill portion at its distal end.

25. The apparatus of claim 23, wherein the second tubular element has a plurality of elongate elements extending from a distal end thereof.

26. The apparatus of claim 25, wherein said elongate members are retractable within lumens formed in a wall of said second tubular element.

27. The apparatus of claim 1, wherein said first anchor member is deployed via self-expansion and said second anchor member is deployed via forced-expansion.

28. The apparatus of claim 27, wherein said first and second anchor members are disposed within a first tubular element during introduction into said vessel or duct.

29. The apparatus of claim 28, wherein said first and second anchor members are disposed over a second tubular element having an expansion member disposed within said second anchor member.

30. The apparatus of claim 1, wherein at least one of said first and second anchor members is substantially longer than the other of said first and second anchor members and deployable via forced expansion.

31. The apparatus of claim 30, wherein said first anchor element is deployable via self-expansion and substantially shorter than said second anchor element, and said second anchor element is deployable via self-expansion and substantially longer than said first anchor element and deployable via forced-expansion.

32. The apparatus of claim 31, wherein during introduction into the vessel or duct the first anchor element is retained within a tubular element during introduction and said second anchor element is retained along the exterior of the tubular element.

33. The apparatus of claim 32, wherein said tubular element includes an expansion member for forcibly expanding said second anchor member within said vessel or duct.

34. The apparatus of claim 33, wherein said tubular element may be removed from said vessel or duct following the deployment of said second anchor member, thereby allowing said first anchor member to deploy via self-expansion.

35. A method of manufacturing a graft assembly for introduction into a living body, comprising the steps of:
providing a length of graft conduit having a first end, a second end and a lumen extending therebetween;
coupling a first anchor member to said first end of said graft conduit; and
coupling a second anchor member to said second end of said graft conduit.

36. The method of manufacture of claim 35, wherein said graft conduit comprises a length of bio-compatible synthetic conduit.

37. The method of manufacture of claim 35, wherein the graft conduit comprises a length of autologous blood vessel obtained by one of harvesting said autologous blood vessel from the living body and growing said autologous blood vessel using bio-engineering techniques.

38. The method of manufacture of claim 35, wherein the graft conduit is dimensioned having a length ranging from 5 mm to 50 mm.

39. The method of manufacture of claim 35, wherein the graft conduit is dimensioned having a diameter ranging from 2 mm to 5 mm.

40. The method of manufacture of claim 35, wherein the graft conduit is dimensioned having a wall thickness ranging from 0.01 mm to 0.5 mm.

41. The method of manufacture of claim 35, wherein the anchor members are constructed to provide sufficient rigidity to maintain the first and second ends of said graft conduit open upon deployment within a vessel or duct.

42. The method of manufacture of claim 35, wherein the anchor members are constructed from at least one of stainless steel, a bio-compatible composite, and Nitonol.

43. The method of manufacture of claim 35, wherein the anchor members are dimensioned having a length ranging from 0.5 mm to 50 mm.

44. The method of manufacture of claim 35, wherein the anchor members are dimensioned having an initial diameter ranging from 1 mm to 3 mm.

45. The method of manufacture of claim 35, wherein the anchor members are dimensioned having a diameter upon deployment ranging from 2 mm to 5 mm.

46. The method of manufacture of claim 35, wherein the anchor members are dimensioned having a wall thickness ranging from 0.02 mm to 0.2 mm.

47. The method of manufacture of claim 35, wherein said anchor members are coupled to said graft conduit via coupling elements.

48. The method of manufacture of claim 47, wherein said coupling elements are elastomeric.

49. The method of manufacture of claim 47, wherein the coupling elements are constructed from at least one of silicone and any polymer or composition having contractility characteristics.

50. The method of manufacture of claim 47, wherein the coupling elements are dimensioned having a width when deployed of approximately 0.05 mm and a width prior to deployment of approximately 0.15 mm.

51. The method of manufacture of claim 47, wherein said coupling elements are connected to said anchor members and said graft conduit via at least one of adhesives and ultrasonic welding.

52. A system for deploying a graft conduit within an intraluminal target site, comprising:
a graft conduit having a first anchor member coupled to a first end and a second anchor member coupled to a second end, said first and second anchor members being capable of deployment via forced-expansion; and
a tubular member having at least one expandable member for selectively expanding said first and second anchor members and thereby opening said first and second ends of said graft conduit within said intraluminal target site.

53. A system for deploying a graft conduit within an intraluminal target site, comprising:
a graft conduit having a first anchor member coupled to a first end and a second anchor member coupled to a second end, said first and second anchor members being capable of deployment via self-expansion; and
a tubular member disposed over said first and second anchor members during introduction into said intraluminal target site and removable thereafter to permit said first and second anchor members to expand and thereby open said first and second ends of said graft conduit within said intraluminal target site.

54. A system for deploying a graft conduit within an intraluminal target site, comprising:
a graft conduit having first and second ends, a first anchor member coupled to said first end, a second anchor member coupled to said second end, said first anchor member being capable of deployment via self-expansion and said second anchor member being capable of deployment via forced-expansion; and
a tabular member having at least one expandable member, said tubular member disposed over at least one of said fist and second anchor members during introduction into said intraluminal target site and removable thereafter to permit said at least one of said first and second anchor members to expand, and said expandable member for selectively expanding said second anchor member, to collectively open said first and second ends of said graft conduit within said intraluminal target site.

55. An apparatus for lining an intraluminal target site, comprising:
a lining having a first anchor member and a second anchor member, said first anchor member being self-expanding and said second anchor member being forcibly-expanding; and
a delivery element having at least one expandable member and at least one restraining member, said at least one restraining member for initially restraining said first anchor member and thereafter being removed to deploy said first anchor member, and said at least one expandable member for expanding to forcibly deploy said second anchor member.

56. The apparatus of claim 55, wherein said second expandable member comprises a balloon dimensioned to be selectively inflated to deploy said second anchor member.

57. The apparatus of claim 55, wherein said delivery element comprises an elongated tubular member having an inner lumen capable of receiving said first anchor member in a constrained state.

58. The apparatus of claim 57, wherein said elongated tubular member is equipped having said at least one expandable member disposed along a portion of an exterior surface thereof.

59. The apparatus of claim 58, wherein at least a portion of said second anchor member is disposed along at least a portion of an exterior surface of said expandable member.

60. The apparatus of claim 55, wherein said second anchor member is substantially longer than said first anchor member.
